(19) United States
(12) Patent Application Publication
(10) Pub. No.: US 2021/0177289 A1
(43) Pub. Date: Jun. 17, 2021

(54) Heart Rate Detection System and Heart Rate Detection Method

(71) Applicant: NATIONAL SUN YAT-SEN UNIVERSITY, KAOHSIUNG CITY (TW)

(72) Inventors: Jau-Sheng Wang, Kaohsiung City (TW); Ting-Chia Ho, Kaohsiung City (TW); Yu-Chiao Sue, Kaohsiung City (TW); Jhen-Wei Huang, Kaohsiung City (TW)

(21) Appl. No.: 16/846,685

(22) Filed: Apr. 13, 2020

(30) Foreign Application Priority Data
Dec. 17, 2019 (TW) 108146235

(51) Int. Cl. A61B 5/024; A61B 5/00

(52) U.S. Cl. CPC A61B 5/02444; A61B 5/0097; A61B 2562/0242; A61B 2562/182; A61B 5/7257

(57) ABSTRACT

A heart rate detection system includes a light source outputting a light beam, an acousto-optical sensing element having a crystalline material, and a light analysis module. The crystalline material has an input end, an output end and a sensing end. The input end is connected to the light source. The light beam emits into the input end, passes through the crystalline material, and emits out of the output end. An acoustic wave signal is received by the sensing end and changes a structure of the crystalline material. The light analysis module is connected to the output end and receives and analyzes the light beam that passes through the crystalline material.

### CROSS REFERENCE TO RELATED APPLICATION

The application claims the benefit of Taiwan application serial No. 108146235, filed on Dec. 17, 2019, and the subject matter of which is incorporated herein by reference.

### BACKGROUND OF THE INVENTION

### 1. Field of the Invention

The present invention generally relates to an acoustic detecting and spectrum analyzing technology and, more particularly, to a heart rate detection system and a heart rate detection method that provide a convenient operation and a fast analysis.

### 2. Description of the Related Art

Heart rate has been an important indicator for the health condition of a person. By monitoring the instant heart rate, the load of the heart can be readily obtained from the heart beat per minute. Generally, under the same activity and environment, the lower the heart rate is, the better the health condition is. The heart rate can be measured in an easy way and can even be recorded through a simple instrument such as a wearable device. When such a device is used by an athele, the change in the heart rate of the athele can be analyzed to obtain the exercise condition and the fatigue condition of the athele.

In addition, Heart Rate Variability (HRV) is another important indicator for evaluating the health of the heart. Heart rate variability is the variation in the time interval between heartbeats. Generally, the heart activity can be intensively recorded in a certain period of time through an electrocardiography (ECG) to generate a continuous wave that varies over time. Then, the characteristics of the waves of the electrocardiography such as the locations of the wave groups, the intervals between the waves, and the heights of the peaks, are compared to obtain the abnormal heart rate and even to analyze the balance of the autonomic nerves. However, the conventional measuring process requires that the multiple-lead electrodes be adhered to the skin on various parts of the limbs. In order to avoid signal interference which adversely affects the measuring accuracy, prior to the adhesion of the electrode pads, the clothes and accessories should be removed, and even the skin should be cleaned and the hair should be shaved. Due to this, the conventional heart rate detection process is complex and liable to cause uncomfortable feeling of the subject and to also invade the subject's privacy.

In light of this, it is necessary to improve the conventional heart rate detection technology.

### SUMMARY OF THE INVENTION

It is therefore the objective of this invention to provide a heart rate detection system which omits the use of the electrode pads and enables a convenient and fast detection.

It is another object of the invention to provide a heart rate detection system which is immune to the electromagnetic interference as it detects the heart rate through an optical signal that can be adaptively adjusted according to the electrocardiography.

It is yet another object of the invention to provide a heart rate detection method which is able to assist the diagnosis under the electrocardiography.

It is a further object of the invention to provide a heart rate detection method which is able to proceed with the diagnosis of the heart by comparing the magnitude change in the frequency domain.

As used herein, the term “one” or “an” for describing the number of the elements and members of the present invention is used for convenience, provides the general meaning of the scope of the present invention, and should be interpreted to include one or at least one. Furthermore, unless explicitly indicated otherwise, the concept of a single component also includes the case of plural components.

In an aspect, a heart rate detection system includes a light source outputting a light beam, an acousto-optical sensing element having a crystalline material, and a light analysis module. The crystalline material has an input end, an output end and a sensing end. The input end is connected to the light source. The light beam emits into the input end, passes through the crystalline material, and emits out of the output end. An acoustic wave signal is received by the sensing end and changes a structure of the crystalline material. The light analysis module is connected to the output end and receives and analyzes the light beam that passes through the crystalline material.

In another aspect, a heart rate detection method includes forming a crystalline material as a grating structure having a periodically-changed spacing pattern through an acoustic wave signal generated by a pulse, guiding a light beam into the grating structure of the crystalline material to cause diffraction of the light beam such that the light beam is attenuated at a predetermined wavelength, generating a light-intensity spectrum of the light beam by analyzing the light beam that passes through the grating structure via a light analysis module, determining by the light analysis module a location of the predetermined wavelength and a frequency of the acoustic wave signal corresponding to the predetermined wavelength according to the light-intensity spectrum of the light beam, and generating a periodically-changed signal intensity diagram by the light analysis module depicting a relationship of a light intensity over time for the predetermined wavelength of the light beam. A change in a waveform of the periodically-changed signal intensity diagram corresponds to a change in a waveform of the acoustic wave signal. The grating structure is modulated according to a frequency of the acoustic wave signal.

Based on this, the heart rate detection system and the heart rate detection method according to the present invention, by changing the structure of the crystalline material with an acoustic wave, cause an energy change in the light beam that passes through the crystalline material. This converts the pulsations of the subject from an acoustic-wave form into a spectrum form. In this regard, the analytic operations are performed to obtain the time-domain intensity change diagram which can be corresponded to a general electrocardiography. By touching the pulse areas of the body with the crystalline material, the heart rate detection result can be obtained in a fast and simple manner without requiring the complex procedures such as the adhesion of the electrode pads and the removal of the clothes and accessories. Advantageously, the operation is convenient and fast, and the system is immune to the electromagnetic interference.

In an example, the light source adjusts and fixes the light beam to a wavelength and a bandwidth. As such, the frequency of the acoustic wave signal to be measured can correspond to a predetermined wavelength of the light beam, increasing the range of measurement and the signal intensity.

In the example, the sensing end of the acousto-optical sensing element is configured to be touched by an area of a superficial artery of a human body, and the acoustic wave signal is pulse. As such, the acousto-optical sensing element can read the pulse, thereby simplifying and facilitating the measurement procedure.

In the example, the crystalline material is fused quartz, high lead glass, tellurium dioxide crystal, lead molybdate crystal or gallium phosphide crystal. As such, the crystalline material can be downsized and does not need to be driven by energy, achieving a pocketable and convenient use.

In the example, the light analysis module generates a light-intensity spectrum according to the light beam. As such, a predetermined wavelength corresponding to the frequency of the acoustic wave can be found by analyzing the light-intensity spectrum, enabling the detection of the heart rate.

In the example, the light analysis module generates a periodically-changed signal intensity diagram according to a predetermined wavelength of the light beam corresponding to a frequency of the acoustic wave signal. As such, a wave diagram corresponding to the electrocardiography is generated, enabling the monitoring of the variation of the heart rate.

In the example, the light analysis module generates a magnitude-frequency distribution diagram according to the periodically-changed signal intensity diagram. As such, the magnitude change can be compared in the frequency domain, achieving a diagnosis of the heart rate.

In the example, the light analysis module analyzes the periodically-changed signal intensity diagram to calculate a heart rate and assist in a diagnosis under electrocardiography. As such, the heart rate can be analyzed from the detected optical signal, attaining a fast detection and avoiding electromagnetic interference.

In the example, the light analysis module performs a Fourier transform on each of a plurality of heart beat periods of the periodically-changed signal intensity diagram to obtain a plurality of magnitude-frequency distribution diagrams, and compares an intensity among the plurality of magnitude-frequency distribution diagrams to assist in a diagnosis of the heart condition. As such, the health condition reflected by each heart beat cycle can be monitored.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 shows a block diagram according to a preferred embodiment of the invention.

FIG. 2 shows a light intensity diagram according to the preferred embodiment of the invention.

FIG. 3 shows an operation of an acousto-optical sensing element according to the preferred embodiment of the invention.

FIG. 4 shows a periodically-changed signal intensity diagram according to the preferred embodiment of the invention.

FIG. 5 shows a magnitude-frequency distribution diagram according to the preferred embodiment of the invention.

In the various figures of the drawings, the same numerals designate the same or similar parts. Furthermore, when the terms “first”, “second”, “third”, “fourth”, “inner”, “outer”, “top”, “bottom”, “front”, “rear” and similar terms are used hereinafter, it should be understood that these terms have reference only to the structure shown in the drawings as it would appear to a person viewing the drawings, and are utilized only to facilitate describing the invention.

### DETAILED DESCRIPTION OF THE INVENTION

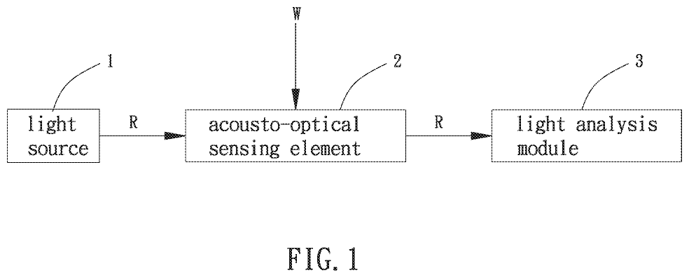

FIG. 1 shows a heart rate detection system according to a preferred embodiment of the invention. The heart rate detection system includes a light source 1, an acousto-optical sensing element 2 connected to the light source 1, and a light analysis module 3 connected to the acousto-optical sensing element 2.

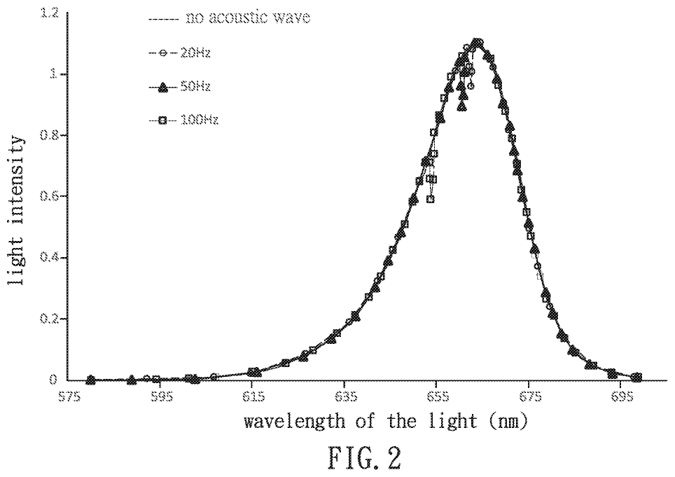

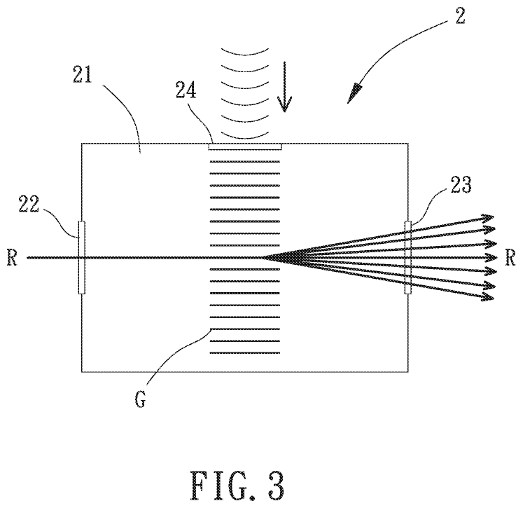

Referring to FIGS. 1 and 2, the light source 1 can be a continuous duty laser and emits a light beam R which may have a narrow bandwidth and high stability and concentration. The light source 1 can control the path of the light beam R and focus on a certain range of the wave length, enabling the light beam R to explore the interaction between the environment and the light. As shown in FIG. 2, the detection can be carried out by analyzing the change in the intensity of the light beam R at a predetermined wavelength. In this embodiment, the spectrum of the light beam R exhibits a normal distribution, in which the central wavelength of the light beam R is 665 nm and the full width at half maximum of the spectrum of the light beam R is 25 nm.

The acousto-optical sensing element 2 may be an acousto-optic modulator (AOM) or a double-clad fiber (DCF). Embedded in the acousto-optical sensing element 2 is a crystalline material 21 having an input end 22, an output end 23 and a sensing end 24 on an outer surface of the crystalline material 21. The input end 22 is connected to the light source 1. As such, the light beam R emits into the input end 22, passes through the crystalline material 21, and emits out of the output end 23. In this regard, the sensing end 24 receives an acoustic wave signal W which affects the structure of the crystalline material 21, forming the crystalline material 21 as a grating structure G having a periodically-changed spacing pattern. The grating structure G diffracts the light beam R passing through the crystalline material 21. Therefore, the acoustic wave signal W indirectly causes an intensity change of the light beam R at the predetermined wavelength of the spectrum. In this embodiment, the crystalline material 21 may be fused quartz, high lead glass, tellurium dioxide crystal, lead molybdate crystal or gallium phosphide crystal. The acoustic wave signal W may be pulse.

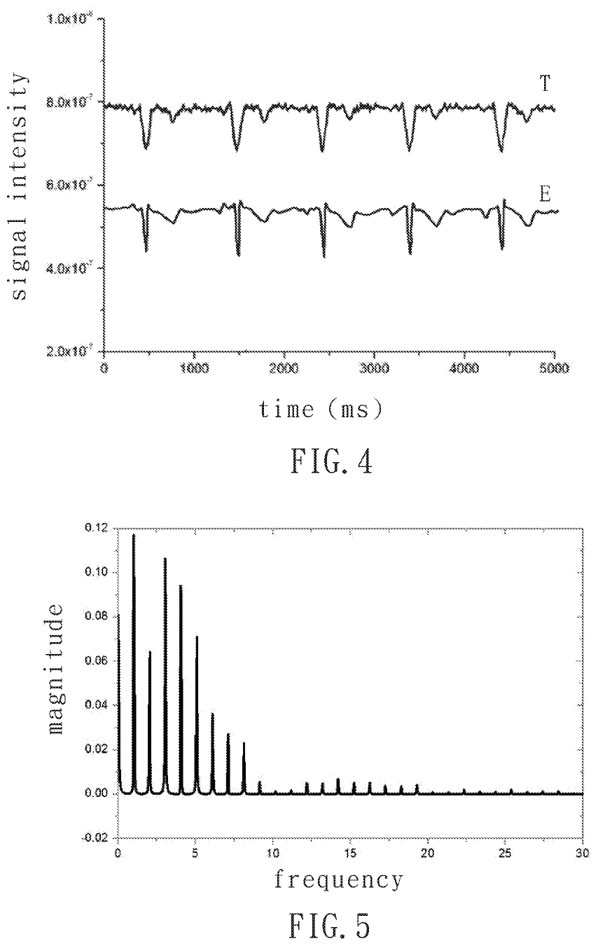

Referring to FIGS. 1 and 5, the light analysis module 3 may be an optical spectrum analyzer (OSA). The light analysis module 3 is connected to the output end 23 to receive the light beam R that passes through the crystalline material 21. As shown in FIG. 2, the light analysis module 3 can display the intensity of the light for a certain frequency range such that a light-intensity spectrum is generated. Furthermore, as shown in FIG. 4, the light analysis module 3 can depict the relationship of the light intensity over time for any wavelength of the light beam R, thereby producing a periodically-changed signal intensity diagram. Next, as shown in FIG. 5, the light analysis module 3 can convert the periodically-changed signal intensity diagram from time domain to frequency domain to obtain a magnitude-frequency distribution diagram.

Based on the above structure, the heart rate detection system according to the invention touches an area of a superficial artery of the subject through the sensing end 24 of the acousto-optical sensing element 2, such as the preauricular region, the neck, the left chest, the fossa cubiti or the wrist. Thus, the crystalline material 21 of the acousto-optical sensing element 2 is modulated according to the frequency of the acoustic wave signal W. In this regard, the light beam R that passes through the crystalline material 21 is diffracted, attenuating the intensity of the light beam R at a predetermined wavelength corresponding to the frequency of the acoustic wave signal W. As shown in FIG. 2, it can be known from the light-intensity spectrum of the light analysis module 3 that the intensity of the light beam R is attenuated at different wavelengths (663 nm, 661 nm, 654 nm) as the light beam R is respectively affected by different frequencies (20 Hz, 50 Hz, 100 Hz) of the acoustic wave signal W. Moreover, as shown in FIG. 4, the light analysis module 3 can, based on the wavelength of the light beam R corresponding to the pulse frequency of the subject, produce a detected optical signal T that varies over time and is depicted as the periodically-changed signal intensity diagram. Then, the light analysis module 3 compares the detected optical signal T with an electrocardiography E of the subject on the time axis, such that the locations of the positive and negative peaks of the electrocardiography E, as well as the interval therebetween, can be identified from the optical signal T. Furthermore, as shown in FIG. 5, the light analysis module 3 performs a Fourier transform on each of the heart beat periods of the periodically-changed signal intensity diagram to obtain a plurality of magnitude-frequency distribution diagrams. As such, each heart beat condition can be analyzed by comparing the changes in magnitude.

The heart rate detection method according to the invention includes forming the crystalline material 21 as the grating structure G (having the periodically-changed spacing pattern) through the acoustic wave signal W generated by the pulse of the subject, guiding the light beam R into the grating structure G of the crystalline material 21 to cause diffraction of the light beam R such that the light beam R is attenuated at the predetermined wavelength, analyzing the intensity change of the light beam R at different wavelengths by the light analysis module 3 to thereby generate the light-intensity spectrum of the light beam R, and determining the location of the predetermined wavelength and a frequency of the acoustic wave signal W corresponding to the predetermined wavelength based on the light-intensity spectrum. The grating structure G is modulated by the frequency of the acoustic wave signal W.

For the predetermined wavelength of the light beam R, the light analysis module 3 depicts the relationship of the light intensity over time to thereby produce the periodically-changed signal intensity diagram. The change in the waveform of the periodically-changed signal intensity diagram corresponds to the change in the waveform of the acoustic wave signal W, thereby calculating the heart rate of the subject to assist in diagnosis of the electrocardiography.

Furthermore, the light analysis module 3 can perform a Fourier transform on each of the heart beat periods of the periodically-changed signal intensity diagram to obtain a plurality of magnitude-frequency distribution diagrams. As such, each heart beat condition can be analyzed by comparing the magnitudes of the plurality of magnitude-frequency distribution diagrams. As compared with the conventional diagnosis which observes the changes in the waveform of the periodically-changed signal intensity diagram, the present invention provides a simple mechanism to detect the heart rate and to monitor the health condition.

In conclusion, the heart rate detection system and the heart rate detection method according to the present invention, by changing the structure of the crystalline material with an acoustic wave, cause an energy change in the light beam that passes through the crystalline material. This converts the pulsations of the subject from an acoustic-wave form into a spectrum form. In this regard, the analytic operations are performed to obtain the time-domain intensity change diagram which can be corresponded to a general electrocardiography. By touching the pulse areas of the body with the crystalline material, the heart rate detection result can be obtained in a fast and simple manner without requiring the complex procedures such as the adhesion of the electrode pads and the removal of the clothes and accessories. Advantageously, the operation is convenient and fast, and the system is immune to the electromagnetic interference.

Although the invention has been described in detail with reference to its presently preferable embodiments, it will be understood by one of ordinary skill in the art that various modifications can be made without departing from the spirit and the scope of the invention, as set forth in the appended claims.

What is claimed is:

1. A heart rate detection system comprising:
a light source outputting a light beam;
an acousto-optical sensing element having a crystalline material, wherein the crystalline material has an input end, an output end and a sensing end, wherein the input end is connected to the light source, wherein the light beam emits into the input end, passes through the crystalline material, and emits out of the output end, wherein an acoustic wave signal is received by the sensing end and changes a structure of the crystalline material; and
a light analysis module connected to the output end and receiving and analyzing the light beam that passes through the crystalline material.

2. The heart rate detection system as claimed in claim 1, wherein the light source adjusts and fixes the light beam to a wavelength and a bandwidth.

3. The heart rate detection system as claimed in claim 1, wherein the sensing end of the acousto-optical sensing element is configured to be touched by an area of a superficial artery of a human body, and wherein the acoustic wave signal is pulse.

4. The heart rate detection system as claimed in claim 1, wherein the crystalline material is fused quartz, high lead glass, tellurium dioxide crystal, lead molybdate crystal or gallium phosphide crystal.

5. The heart rate detection system as claimed in claim 1, wherein the light analysis module generates a light-intensity spectrum according to the light beam.

6. The heart rate detection system as claimed in claim 1, wherein the light analysis module generates a periodically-changed signal intensity diagram according to a predetermined wavelength of the light beam corresponding to a frequency of the acoustic wave signal.

7. The heart rate detection system as claimed in claim 6, wherein the light analysis module generates a magnitude-frequency distribution diagram according to the periodically-changed signal intensity diagram.

8. A heart rate detection method comprising:
forming a crystalline material as a grating structure having a periodically-changed spacing pattern through an acoustic wave signal generated by a pulse, wherein the grating structure is modulated according to a frequency of the acoustic wave signal;
guiding a light beam into the grating structure of the crystalline material to cause diffraction of the light beam such that the light beam is attenuated at a predetermined wavelength;
generating a light-intensity spectrum of the light beam by analyzing the light beam that passes through the grating structure via a light analysis module;
determining a location of the predetermined wavelength and a frequency of the acoustic wave signal corresponding to the predetermined wavelength according to the light-intensity spectrum of the light beam, as performed by the light analysis module; and
generating a periodically-changed signal intensity diagram by the light analysis module depicting a relationship of a light intensity over time for the predetermined wavelength of the light beam, wherein a change in a waveform of the periodically-changed signal intensity diagram corresponds to a change in a waveform of the acoustic wave signal.

9. The heart rate detection method as claimed in claim 8, further comprising analyzing the periodically-changed signal intensity diagram by the light analysis module to calculate a heart rate and assist in a diagnosis under electrocardiography.

10. The heart rate detection method as claimed in claim 8, further comprising:
performing a Fourier transform on each of a plurality of heart beat periods of the periodically-changed signal intensity diagram to obtain a plurality of magnitude-frequency distribution diagrams, as executed by the light analysis module; and
comparing an intensity among the plurality of magnitude-frequency distribution diagrams by the light analysis module to assist in a diagnosis of the heart condition.
